(19) United States
(12) Patent Application Publication
(10) Pub. No.: US 2013/0131768 A1
(43) Pub. Date: May 23, 2013

(54) IMPLANTABLE COLLECTOR ELECTRODE HAVING A CONNECTION CABLE WITH DIRECTION MARKER, AND SYSTEM

(71) Applicant: Marc Possover, Hagendorn (CH)

(72) Inventor: Marc Possover, Hagendorn (CH)

(21) Appl. No.: 13/809,965

(22) PCT Filed: May 25, 2011
(86) PCT No.: PCT/EP11/58570

§ 371 Date: Jan. 14, 2013

(30) Foreign Application Priority Data
May 26, 2010 (DE) 10 2010 021 512.0

(51) Int. Cl. A61N 1/05

(52) U.S. Cl. CPC A61N 1/05
USPC 607/116

(57) ABSTRACT

The invention relates to a wire-shaped collector electrode (10), which can be laparascopically implanted into the pelvis and through the abdominal wall of a human body and which is arranged on the end side on a connection cable (13) that has an outside surface (20), for the neurostimulation of nerves. Said collector electrode comprises several outer segment electrodes (1-8) which can be contacted individually and/or in groups and are disposed axially one behind the other in the direction of the longitudinal extension of the collector electrode (12), wherein an insulating section (101-107) for the electrical insulation of the two respective adjacent outer segment electrodes is disposed axially between said respective two adjacent outer segment electrodes (1, 2; 2, 3; 3, 4; 4, 5; 5, 6; 6, 7; 7, 8). According to the invention, a visually perceptible direction marker (21) is provided on the outside surface (20) of the connection cable, at least in a cable section which is spaced apart from the axial ends (14, 15) of the connection cable and has an axial extension of at least 10 cm and/or 15% of the total length of the connection cable, said direction marker indicating the orientation of the connection cable to the operator.

### BACKGROUND OF THE INVENTION

The invention relates to a wire-shaped collector electrode, which can be implanted by laparoscopy through the abdominal wall into the small pelvis of the human body, is arranged at one end on a connection cable having an outer surface and is used for neurostimulation of nerves, said collector electrode comprising several outer segment electrodes which can be contacted individually or in groups and are arranged axially one after another in the direction of the longitudinal extent of the collector electrode, wherein an insulating section is arranged between in each case two adjacent outer segment electrodes and permits the electrical insulation of the respective two adjacent outer segment electrodes.

From US 2007/0198065 A1 filed by the applicant, it is known to provide neurostimulation of nerves (e.g. plexus sacralis, nervus ischiadicus, nervus pudendus) in the small pelvis by using collector electrodes with eight ring-shaped outer segment electrodes that are spaced axially apart from one another, which collector electrodes are usually implanted by laparoscopy. The collector electrode is connected by a connection cable to a pacemaker, which acts on the collector electrode with a stimulation pattern and in doing so controls the outer segment electrodes individually, in order to selectively stimulate the desired nerve. In the implantation of the pacemaker, it is often necessary for the operator to pull the connection cable in the direction of the axial end having the collector electrode. It can happen that the operator pulls the connection cable in the direction of the pacemaker instead of in the direction of the collector electrode and thus shifts the latter away from the nerve to be stimulated, which then entails awkward repositioning of the collector electrode.

US 2009/0248124 A1 discloses a non-wire-shaped collector electrode for implantation in the human body, wherein the connection cable of the electrode is provided, within a short axial section, with a marker so as to be able to identify the electrode and/or to assign the electrode to the correct connection channel of the pacemaker. In the known electrode, the problem of directional orientation does not arise, since the comparatively broad electrode cannot be shifted out of position by pulling on the connection cable.

US 2010/0030298 A1 discloses an implantable electrode having a marker with which the rotary orientation of the connection for contacting a pacemaker can be determined.

### SUMMARY OF THE INVENTION

Proceeding from the aforementioned prior art, the object of the invention is to propose a collector electrode which has a connection cable, and with which an inadvertent shifting of the collector electrode away from the nerve to be stimulated can be reliably avoided during a surgical intervention. The object is also to make available a system comprising a correspondingly improved collector electrode which is connected by the connection cable to a pacemaker for applying a stimulation pattern to the collector electrode.

In an implantable collector electrode of the type in question, particularly a collector electrode implantable by laparoscopy, this object is achieved by the fact that a direction marker perceptible by sight and/or by touch is provided on the outer surface of the connection cable, at least in an axial section which is spaced apart from the axial ends of the connection cable, said direction marker indicating the orientation of the connection cable to the operator.

As regards the system, the object is achieved by the combination of a collector electrode, designed according to the concept of the invention, together with a pacemaker.

Advantageous additional developments of the invention are also set forth herein. The scope of the invention covers all combinations of at least two of the features disclosed in the description, the claims and/or the figures.

In order to avoid repetition, features that are disclosed in relation to the device are also to be understood as having been disclosed and able to be claimed in relation to the method. Likewise, features that are disclosed in relation to the method are to be understood as having been disclosed and able to be claimed in relation to the device.

The invention is based on the concept of avoiding inadvertent shifting of the collector electrode during implantation of the pacemaker, by providing a direction marker, perceptible by sight and/or by touch, on the outer surface of the connection cable, at least in an axial section which is spaced apart from the axial ends of the connection cable, said direction marker indicating the orientation of the connection cable to the operator. The orientation of the connection cable is to be understood as the direction in which, on the basis of the direction marker, the collector electrode or the axial end directed away from the collector electrode is located. The direction marker thus has the function of providing the operator with information concerning the orientation of the connection cable, such that the operator can see, from the axial section observed, in which direction the connection cable is to be pulled, without this resulting in undesired shifting of the collector electrode at the end away from the nerve. The feature “at least in an axial section which is spaced apart from the axial ends of the connection cable” is to be understood as meaning that the orientation information is intended to be recognizable to the operator in an area spaced apart from the axial ends. Of course, the axial section having the direction marker can also extend as far as at least one of the two axial ends of the connection cable, although the direction marker must at least also be provided in an area spaced apart from the ends. It is important that the operator obtains the orientation information without having to see one of the two axial ends, particularly since these ends are located in regions of the body that are not visible, that are concealed or that are not exposed.

The direction marker can be designed to be perceptible by sight, for example by suitable printing on the outer surface. If appropriate, the marker can also be designed to be perceptible by touch. A direction marker perceptible by touch can be obtained by a raised and/or recessed formation of the direction marker.

The invention is based on the concept that the direction marker is provided over a (relatively long) cable section of at least 10 cm in length and/or of at least 15% of the total length of the connection cable and is designed in such a way that every 2 cm, preferably every 1.5 cm, more preferably every 1.0 cm, very particularly preferably every 0.5 cm, of this cable section is enough to allow the operator to visually establish the orientation of the connection cable (without further optical aids such as magnifying lenses). To put it another way, provision is made according to the invention that the axial extent of the cable section with direction marker measures at least 10 cm, preferably at least 15 cm, more preferably at least 20 cm, very particularly preferably at least 25 cm, and that the orientation of the connection cable can be read off at any desired section of this at least 10 cm long cable section which (the section) has a length of at most 2 cm, preferably at most 1.5 cm, more preferably 1.0 cm, very particularly preferably 0.5 cm. Of course, the preselected section of the cable section provided with the direction marker can also be larger. However, according to the invention, it is sufficient to have an axial extent of 2 cm or less, very particularly preferably of 0.5 cm or less.

The solution according to the invention is therefore in two steps. First, the cable section with the direction marker must be sufficiently long (according to the invention at least 10 cm and/or 15% of the total length of the connection cable), and, second, the direction marker must be so configured, for example by a suitable number of visual markers per centimeter, that the orientation can be read off at each section of this cable section if the section has a longitudinal extent of 2 cm (or more), preferably 1.5 cm (or more), preferably 1 cm or more, very particularly preferably 0.5 cm (or more). With a typical connection cable length of 60 cm, 15% of the total longitudinal extent corresponds to approximately the at least 10 cm.

In the case where the axial extent of the cable section provided with the direction marker is smaller than the axial extent (total axial extent) of the connection cable, it is particularly expedient if most (more than 50%, preferably more than 60%) of the axial extent of the cable section with direction marker is then situated in the proximal section (toward the pacemaker) of the connection cable.

The total axial extent of the connection cable is understood as the axial extent between the collector electrode at the end and the contact or connection area for contacting the pacemaker.

As has already been mentioned, the collector electrode designed according to the concept of the invention serves for neurostimulation of endopelvic nerve sections in the small pelvis, by means of a pacemaker applying a defined stimulation pattern to the collector electrode or the outer segment electrodes of the collector electrode. The stimulation pattern is preferably chosen according to the indication that is to be treated. The collector electrode designed according to the concept of the invention can alternatively also be used as a sensor for detecting nerve impulses. The collector electrode designed according to the concept of the invention is preferably suitable for use in the following indications:
Neurogenic or non-neurogenic hyperactivity of the bladder (sacral and pudendal nerve stimulation)
Neurogenic or non-neurogenic, myogenic hypotonia/atonia of the bladder
Pudendal block (in paraplegia)
Voiding of the bladder/bowels in hyperactivity deblockade (in paraplegia)
Spasticity in the lower extremities, particularly in multiple sclerosis, polyneuropathy, quadriplegia/paraplegia, etc. (sciatic nerve stimulation)
Erectile and sexual problems, loss of erection, inability to ejaculate, premature ejaculation (stimulation of sciatic nerve root/stimulation of pudendal nerve)
Inability to achieve orgasm in females
Neurogenic and non-non-neurogenic urinary/rectal incontinence (stimulation of pudendal nerve)
Chronic constipation
Various pathologies and symptoms (simultaneous stimulation of various (at least two) nerves)
Pudendal neuralgia=>sciatic nerve neuralgia

In addition to restoration of bladder and/or bowel function, deambulation, in particular by stimulation of the sciatic nerve (cf. illustrative embodiment according to FIG. 5) or of at least one sacral nerve root or all the sacral nerve roots (cf. illustrative embodiment according to FIG. 6), the collector electrode designed according to the concept of the invention is suitable for use in further indications listed below:
Therapy of neuropathic pain of the lower extremities (Sudeck's syndrome, stump and phantom pain after amputation, polyneuropathy), in particular through stimulation of the afferent fibers of the sciatic nerve.
Control of spasticity of the lower extremities, in particular from spinal cord injuries or multiple sclerosis.
Muscle build-up in the lower extremities, particularly in the buttocks for prevention of decubitus ulcers in paraplegic patients. Here, the approach followed is to generate as much tissue as possible in an area between the wheelchair and the bone, in order to minimize decubitus ulcers. Muscle contraction is indicated by stimulation of the efferent fibers of the sciatic nerve, as a result of which muscle mass and strength are built up. By stimulation of the sympathetic fibers of the sciatic nerve, a peripheral vasodilation can. This not only serves to prevent decubitus ulcers but can also be used to treat decubitus ulcers.
The treatment of blood pressure problems in paraplegic patients, particularly in quadriplegics. To this end, it is advantageous to stimulate the sciatic nerve for the purpose of controlling the blood control of the lower extremities, which influence the general blood pressure.
Therapy of osteoporosis of the lower extremities in paraplegic patients. The blood supply to the bones is improved by stimulation of the sciatic nerve, particularly of the sympathetic fibers of the nerve. This serves on the one hand to prevent osteoporosis and also as therapy of osteoporosis.

The collector electrode is preferably arranged at an axial end of the connection cable or is formed by an axial end area of the connection cable. It is particularly preferable if the collector electrode has a wire-shaped design. Wire-shaped is to be understood here as an elongate, for example rod-shaped, rigid or, alternatively, possibly deformable configuration.

According to the invention, provision is made that the collector electrode has a wire-shaped design. Wire-shaped is to be understood here as an elongate, for example rod-shaped, rigid or, alternatively, possibly deformable configuration.

The axial extent of the in particular wire-shaped collector electrode is preferably chosen from a value range of between approximately 45 mm and approximately 65 mm. The axial extent is particularly preferably approximately 57 mm. The length dimension relates here to the distance between the opposite axial ends of the outer segment electrodes farthest from each other. It is particularly expedient if the diameter of the collector electrode, which is preferably at least approximately cylindrical, in particular circularly cylindrical, in contour is chosen from a value range of between 0.5 mm and 2 mm, very particularly preferably from a value range of between 0.8 mm and 1.2 mm. The diameter is still more preferably approximately 1 mm.

A very particularly preferred embodiment of the collector electrode is one in which the direction marker consisting of a multiplicity of individual symbols, or the cable section having the direction marker, has, in a section spaced apart by at least 10 cm, preferably at least 15 cm from the proximal end (toward the pacemaker), an axial extent of at least 10 cm, preferably at least 15 cm, more preferably at least 20 cm, very particularly preferably at least 25 cm, in order to be able to determine the orientation in an area of the connection cable spaced apart relatively far from the pacemaker, wherein at most every 2 cm, preferably at most every 1.5 cm, more preferably at most every 1.0 cm, very particularly preferably at most every 0.5 cm, of this cable section is enough to allow the orientation of the connection cable to be determined visually without optical aids.

As has already been indicated at the outset, the at least one axial section having the direction marker does not necessarily have to end at a distance from one or both axial ends of the connection cable, and instead, if so desired, it can be continued as far as at least one of the axial ends of the connection cable.

It is important, however, that the orientation of the connection cable can be read off from the direction marker at least in one area spaced apart from the axial ends.

In an alternative embodiment, the at least one axial section provided with a direction marker ends at an axial distance in front of at least one of the two axial ends of the connection cable.

This distance is preferably less than 25% of the total longitudinal extent of the connection cable, more preferably less than 15%.

There are various possibilities regarding the specific configuration of the direction marker. For example, it is possible that the direction marker is formed by a multiplicity of symbols that are arranged axially in succession, are spaced apart from one another or connected to one another, and are perceptible by sight and/or touch. For example, these can be arrow symbols that point in one of the two axial directions, in order thereby to indicate the position of the collector electrode or the position of the pacemaker relative to the arrow symbol. It is very particularly preferable if the symbols are arranged in a row. It is very particularly preferable if the symbols are spaced axially apart from one another. If necessary, several rows of symbols spaced apart in the circumferential direction can be provided. The at least one row of symbols preferably extends, at least more or less, along the entire axial extent of the connection cable.

It is essential to provide a sufficient number of symbols per unit of length, so as to ensure that the orientation can be read off at each section with a length of 2 cm, 1.5 cm, 1.0 cm, preferably 0.5 cm, of the at least 10 cm long cable section.

Particularly if no arrow symbols are used to indicate orientation, the orientation can be signaled by the fact that a geometric feature, for example an axial extent and/or a circumferential extent of the symbols, varies from symbol to symbol or from symbol group to symbol group, where each symbol group comprises at least two symbols. In other words, a geometric dimension, for example, decreases or increases from symbol to symbol toward one of the two axial ends, in order thereby to provide the orientation information upon simultaneous observation of at least two symbols or symbol groups.

In an alternative embodiment, particularly when arrow symbols are used, the symbols arranged one after another can be of identical design.

In another alternative embodiment, the direction marker can comprise a single symbol or a small number, e.g. only two, three or four in total, of axially adjacent symbols, wherein the information concerning direction or orientation can be read off from the change in a geometric dimension of the symbol or symbols. For example, the circumferential extent of the symbol can decrease toward one axial end, resulting, for example, in an extremely elongate arrow symbol.

A very particularly preferred embodiment of the collector electrode is one in which it comprises at least five, preferably at least six, very particularly preferably at least seven, still more preferably eight, outer segment electrodes that are spaced apart in the axial direction and are preferably ring-shaped.

An embodiment in which the collector electrode is arranged at one end on the connection cable is particularly expedient.

This means that the actual collector electrode, i.e. the arrangement of outer segment electrodes, closes off an axial end of the connection cable or forms an end section of the connection cable. Thus, the collector electrode is not situated at just any axial position on the connection cable, but expressly at an axial end, so as to be able to position the collector electrode optimally, in particular by grasping the connection cable. The collector electrode thus forms the end continuation of the connection cable or of the connection cable end, resulting in a wire-shaped configuration of the collector electrode/connection cable arrangement. The diameter of the connection cable preferably corresponds, at least more or less (±10%), to the diameter of the collector electrode preferably formed by the end section of the connection cable.

There are various possibilities regarding the configuration of the insulating sections and/or of the outer segment electrodes. For example, these can extend only around sections of the circumference. However, it is particularly preferable if the insulating sections and/or the outer segment electrodes are ring segments closed all the way round the circumference.

The axial extent of the outer segment electrodes is preferably chosen from a value range of between approximately 1 mm and approximately 5 mm. The axial extent is preferably approximately 3 mm. The axial extent of at least one of the insulating sections is preferably chosen from a value range of between 2 mm and 7 mm. The axial extent is preferably 3 mm or 6 mm.

The invention also leads to a system for neurostimulation of nerves, comprising a collector electrode as described above with connection cable, wherein the outer segment electrodes of the collector electrode can be electrically controlled individually and/or in groups by an in particular eight-channel pacemaker, wherein the pacemaker is arranged at an axial end of the connection cable directed away from the outer segment electrodes.

### BRIEF DESCRIPTION OF THE DRAWINGS

Further advantages, features and details of the invention will become clear from the following description of preferred illustrative embodiments and by reference to the drawings, in which:

FIG. 1 shows a system comprising a pacemaker connected by a connection cable to a collector electrode, wherein a row of arrow symbols arranged axially alongside one another is provided as direction marker on the connection cable,

FIG. 2 shows a system of analogous design to the system according to FIG. 1, with the difference that two rows of symbols spaced apart from each other in the circumferential direction are provided as direction marker,

FIG. 3 shows an alternative system comprising a collector electrode with connection cable and a pacemaker, wherein a repeating arrangement of two different symbols is provided as direction marker, and

FIG. 4 shows a system in which a multiplicity of symbols arranged one after another are provided as direction marker, which symbols are designed as ring elements in the illustrative embodiment shown, wherein the axial extent of the symbols decreases in the direction of the collector electrode.

### DETAILED DESCRIPTION

Elements that are the same and elements that have the same function are indicated by the same reference signs in the figures.

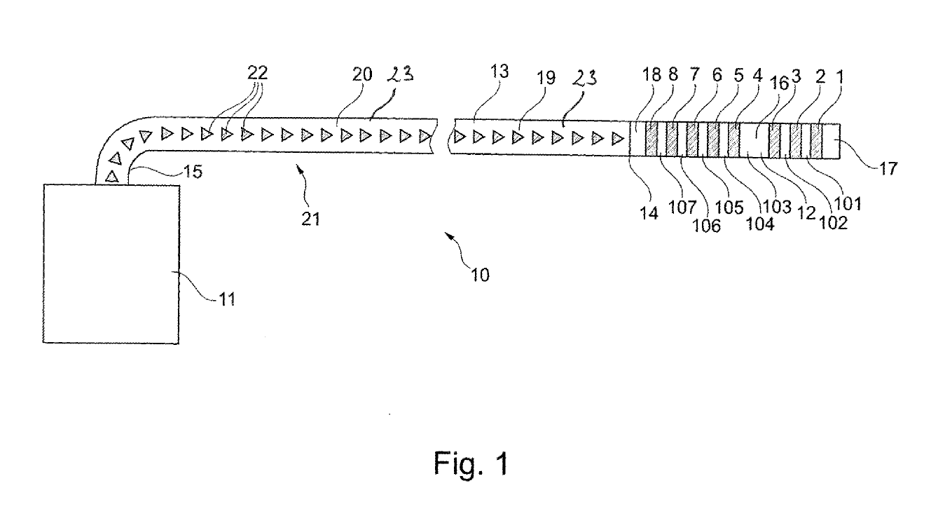

FIG. 1 shows a highly schematic representation of a system 10 for neurostimulation of nerves. The system 10 comprises an implantable pacemaker 11, which is shown much too small in the drawing compared to a collector electrode 12 likewise included in the system. The pacemaker 11 is connected to the collector electrode 12 by a connection cable 13 which, as will be explained in more detail below, comprises a plurality of leads electrically insulated from one another. The collector electrode 12 adjoins the connection cable 13 axially, such that connection cable 13 and collector electrode 12 have a substantially wire-shaped configuration. The connection cable 13 comprises a first and distal end 14 at a transition to the collector electrode 12. Directed away from the distal and first axial end 14, the connection cable 13 has a second axial end 15, which adjoins the pacemaker 11.

In the preferred illustrative embodiment shown, the substantially cylindrical surface 16 of the collector electrode 12 comprises a total of eight outer segment electrodes 1 to 8, counting upward from the free end (situated on the right in the plane of the drawing) of the collector electrode 12. The outer segment electrodes 1 to 8 are individually controllable by means of the pacemaker 11 and are electrically insulated from one another. For this purpose, insulating sections 101 to 107 (likewise counting upward from the free end of the collector electrode 12) are situated between in each case two of the outer segment electrodes 1, 2; 2, 3; 3, 4; 4, 5; 5, 6; 6, 7; 7, 8 arranged axially one after another. It will be seen from FIG. 1 that the axial extent of the third insulating section 103, counting from the free end of the collector electrode 12, is greater and, in the illustrative embodiment shown, approximately twice as great as the axial extent of all the other insulating sections 101, 102 and 104 to 107. These other insulating sections 101, 102 and 104 to 107 have the same axial extent, approximately 3 mm in this illustrative embodiment. It will also be seen from FIG. 1 that the circumferentially closed outer segment electrodes 1 to 8, shaped as ring segments, are all of the same size, and they all have the same axial extent of 3 mm in the illustrative embodiment shown. The axial extent of the entire collector electrode 12 measures 57 mm in the illustrative embodiment shown. The diameter measures 1 mm.

It will also be seen from FIG. 1 that, to the right of the first outer segment electrode in the plane of the drawing, a first insulating end section 17 is provided, which is spaced apart from and faces away from a second end section 18 that forms the end directed toward the first end 14 of the connection electrode 13.

Alternatively, an embodiment is conceivable in which all the insulating sections 101 to 107 are of the same size. It is also possible that it is not the third insulating section 103, but another insulating section 101, 102 or 104 to 107, that is larger than the other insulating sections.

In the illustrative embodiment shown, the axial extent of the flexible, wire-shaped connection cable measures 20 mm. Each outer segment electrode 1 to 8 is individually contacted by an electrically insulated (control) lead, not shown for reasons of clarity, wherein all the leads are guided out from the collector electrode 12, specifically at the end of the collector electrode 12 directed toward the connection cable 13. Up to there, the leads are guided in the interior of the collector electrode 12 at a radial distance from the circumferential wall of the collector electrode. The leads join up to form the single connection cable 13 provided with a jacket 19 and used to contact the pacemaker 11.

The outer surface 20 of the connection cable 13 is provided with a direction marker 21, which indicates the orientation of the connection cable 13, that is to say the relative position of the axial ends 14, 15. In the illustrative embodiment shown, the direction marker 21 is formed by a multiplicity of in this case arrow-shaped symbols 22 arranged one after another in the axial direction, the tip of the arrows pointing in the direction of the collector electrode 12 in the illustrative embodiment shown. Alternatively, the tips of the arrows can of course be designed or arranged pointing in the direction of the pacemaker 11.

The operator simply has to know in which direction the arrow symbols point. In the illustrative embodiment shown, the symbols 22 arranged in a row extend, at least more or less, along the entire longitudinal extent of the connection cable 13 and are therefore also present in axial sections arranged at an axial distance from the ends 14, 15. The cable section provided with the direction marker 21 has a longitudinal extent of well over 10 cm and is indicated by reference sign 23. In the illustrative embodiment shown, the operator can read off the orientation of the connection cable 13 at any desired axial section of the cable section 23 having a length of at least 0.5 cm.

The following illustrative embodiments correspond substantially to the above-described illustrative embodiment of a system 10 as shown in FIG. 1, and therefore, in order to avoid repetition, it is essentially only the differences that will be discussed. As regards the common features, reference is made to the preceding figure description. In all of the illustrative embodiments, the cable section 23 with the direction marker is very long in relation to the total length of the connection cable, as is advantageous. In principle, it is sufficient for the cable section 29 to have a minimum length of 10 cm, preferably 15 cm, more preferably 20 cm. In all of the illustrative embodiments, which are not reproduced true to scale, it is ensured that the orientation can be read off at any desired 0.5 cm sections of the cable section 23.

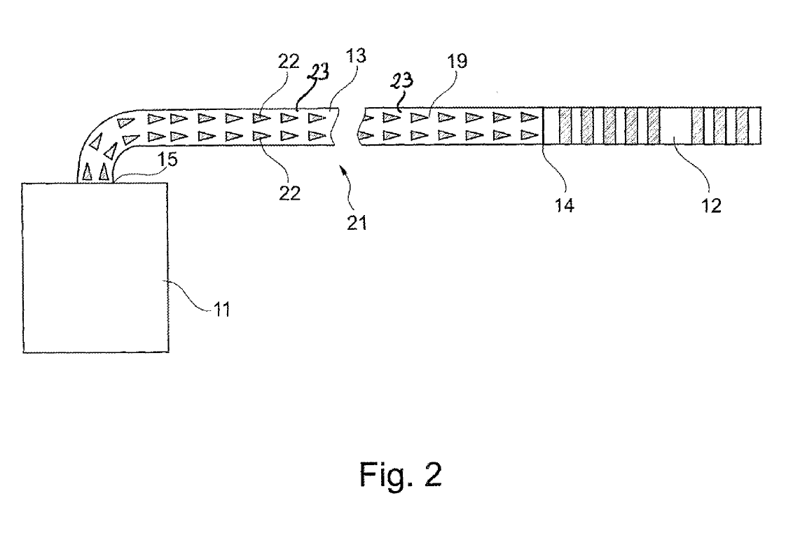

In FIG. 2, in contrast to the illustrative embodiment according to FIG. 1, the direction marker 21 comprises several rows of in this case arrow-shaped symbols 22 spaced apart from each other in the circumferential direction. As in the illustrative embodiment according to FIG. 1, the symbols 22 can be designed, for example, so as to be perceptible exclusively by sight.

It is preferable for them to be perceptible by a combination of sight and touch. For this purpose, the arrow symbols can be raised, for example, or designed as depressions in the jacket 19.

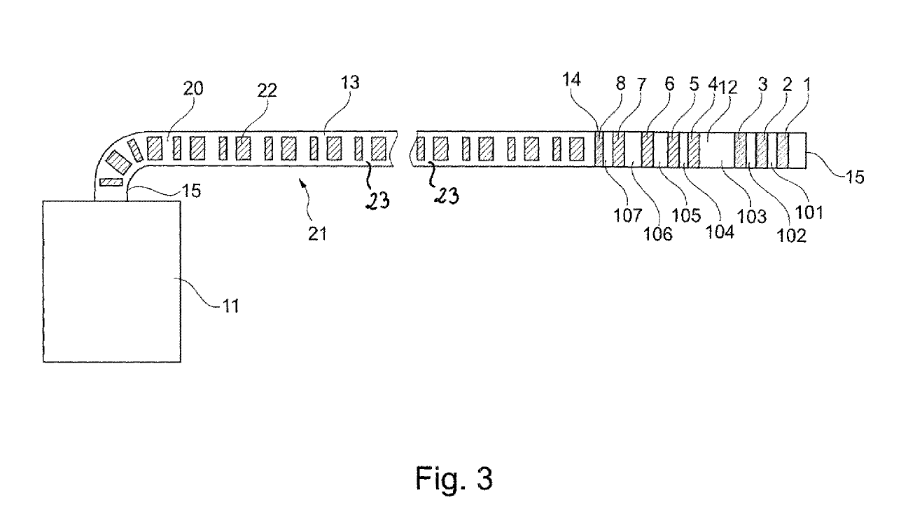

In the illustrative embodiment according to FIG. 3, the direction marker 21 on the outer surface 20 of the connection cable 13 comprises a repeated arrangement of symbols 22 which are each arranged in pairs and, through their arrangement relative to each other, embody directional information. In the illustrative embodiment shown, each pair of symbols comprises two rectangles of different sizes that differ in terms of their axial extent, wherein the distance between the symbols (small rectangle, large rectangle) of a symbol pair is different than the distance between in each case two adjacent symbol pairs. Instead of rectangles, other symbol geometries can also be chosen. Similarly, it is also possible for more than three contiguous symbols to embody directional information. In the illustrative embodiment shown, the distance between two symbols of a symbol pair is smaller than the distance between two symbol pairs. To be able to interpret the directional information, the user merely needs the information that, in the illustrative embodiment in question, the smaller symbols face in the direction of the pacemaker 11 and the larger symbols face in the direction of the collector electrode 12.

In the illustrative embodiment according to FIG. 3, an alternative preferred collector electrode 12 is provided that has a total of eight outer segment electrodes 1 to 8, wherein the insulating sections 101, 102 and 104 between the first and second outer segment electrodes 1, 2, between the second and third outer segment electrodes 2, 3 and between the fourth and fifth outer segment electrodes 4, 5 have a smaller axial extent, namely 3 mm in the illustrative embodiment shown, than the insulating sections 103, 105, 106 and 107 between the fourth and fifth outer segment electrodes 4, 5, between the fifth and sixth outer segment electrodes 5, 6, between the sixth and seventh outer segment electrodes 6, 7, and between the seventh and eighth outer segment electrodes 7, 8. The collector electrode shown in FIG. 3 can also be used with the alternative direction markers 21 of FIGS. 1, 2 and 4. If necessary, it is possible to omit the insulating section to the right of the first outer segment electrode 1 in the illustrative embodiment according to FIG. 3, and also in the other illustrative embodiments. The outer segment electrodes 1 to 8 and the insulating sections 101 to 107 are counted from the free end of the collector electrode 12.

The symbol pairs preferably extend over at least 25% of the total longitudinal extent of the connection cable 13.

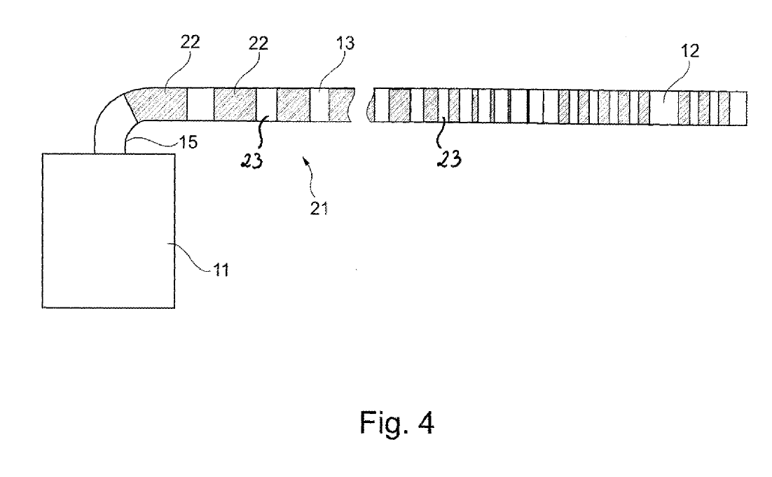

In FIG. 4, the direction marker 21 comprises a multiplicity of successive symbols 22, which are spaced axially apart and, in the illustrative embodiment shown, are designed as ring-shaped symbols. The axial extent of the symbols 22 decreases from symbol to symbol in the direction of the collector electrode 12, such that the operator, by simultaneously observing two adjacent symbols 22, can read off the orientation of the connection cable 13.

All of the symbols shown in FIGS. 1 to 4 can be designed to be perceptible only by sight and/or to be perceptible by touch, for example by designing the symbols as elevations or depressions.

1. A wire-shaped collector electrode (10), which can be implanted by laparoscopy through the abdominal wall into the small pelvis of the human body, is arranged at one end on a connection cable (13) having an outer surface (20) and is used for neurostimulation of nerves, said collector electrode comprising several outer segment electrodes (1-8) which can be contacted individually and/or in groups and are arranged axially one after another in the direction of the longitudinal extent of the collector electrode (12), wherein an insulating section (101-107) is arranged axially between in each case two adjacent outer segment electrodes (1, 2; 2, 3; 3, 4; 4, 5; 5, 6; 6, 7; 7, 8) and permits the electrical insulation of the respective two adjacent outer segment electrodes (1, 2; 2, 3; 3, 4; 4, 5; 5, 6; 6, 7; 7, 8), wherein a visually perceptible direction marker (21) is provided on the outer surface (20) of the connection cable (13), at least in a cable section which is spaced apart from the axial ends (14, 15) of the connection cable (13) and has an axial extent of at least 10 cm and/or at least 15% of the total length of the connection cable (13), said direction marker (21) indicating the orientation of the connection cable (13) to the operator, and wherein the direction marker (21) is designed and arranged in such a way that the identification of the orientation of the connection cable (13) is possible at any desired axial section of the cable section having a maximum axial extent of 2 cm.

2. The collector electrode as claimed in claim 1, wherein the axial extent of the cable section measures at least 15 cm.

3. The collector electrode as claimed in claim 1, wherein the axial extent of the axial section provided with the direction marker is less than the total axial extent of the connection cable, and wherein more than 50%, of the axial extent of the cable section is arranged between an axial connection cable center and the proximal end of the connection cable.

4. The collector electrode as claimed in claim 1, wherein the direction marker (21) ends before at least one of the two axial ends (14, 15) of the connection cable (13) at an axial distance whose longitudinal extent corresponds to 5% to 25% of the total longitudinal extent of the connection cable (13).

5. The collector electrode as claimed in claim 1, wherein the direction marker (21) comprises a multiplicity of symbols (22) which are arranged axially one after another and are perceptible by sight and/or touch.

6. The collector electrode as claimed in claim 5, wherein a geometric feature of the symbols (22) changes from symbol to symbol or from symbol group to symbol group as the distance to one of the axial ends (14, 15) of the connection cable (10) decreases.

7. The collector electrode as claimed in claim 5, wherein at least two of the symbols (22) are of identical design.

8. The collector electrode as claimed in claim 1, wherein the direction marker (21) comprises at least one elongate symbol (22) extending in the axial direction and changing in terms of geometry along its axial extent.

9. A system for neurostimulation of endopelvic sections of nerves in the small pelvis, comprising a collector electrode (12) as claimed in claim 1, of which the outer segment electrodes (1-8) can be electrically controlled individually and/or in groups by a preferably eight-channel pacemaker (11), wherein the pacemaker (11) is arranged on an axial end (15) of the connection cable (13) directed away from the outer segment electrodes (1-8).

10. Use of a collector electrode (12) claim 1, wherein a system (10) as claimed in claim 9 for neurostimulation of endopelvic sections of nerves in the small pelvis.

11. The collector electrode as claimed in claim 1, wherein the desired axial section of the cable section has a maximum axial extent of 1.5 cm.

12. The collector electrode as claimed in claim 1, wherein the desired axial section of the cable section has a maximum axial extent of 1.0 cm.

13. The collector electrode as claimed in claim 1, wherein the desired axial section of the cable section has a maximum axial extent of 0.5 cm.

14. The collector electrode as claimed in claim 2, wherein the axial extent of the cable section measures at least 20 cm.

15. The collector electrode as claimed in claim 2, wherein the axial extent of the cable section measures at least 25 cm.

16. The collector electrode as claimed in claim 3, wherein more than 60% of the axial extent of the cable section is arranged between an axial connection cable center and the proximal end of the connection cable.

17. The collector electrode as claimed in claim 4, wherein the longitudinal extent of the axial distance is 5 to 15% of the total longitudinal extent of the connection case (13).

18. The collector electrode as claimed in claim 5, wherein the multiplicity of symbols (22) are in the shape of directional arrows.

19. The collector electrode as claimed in claim 6, wherein the geometric feature is an axial extent and/or a circumferential extent of the symbols (22).

20. The collector electrode as claimed in claim 7, wherein all of the symbols (22) are of identical design.

21. The collector electrode as claimed in claim 8, wherein the at least one elongate symbol (22) extends over at least 25% of the longitudinal extent of the connection cable (13).
